(19) United States
(12) Patent Application Publication
(10) Pub. No.: US 2026/0083981 A1
(43) Pub. Date: Mar. 26, 2026

(54) A SHIELDED FLEXIBLE BAG FOR DELIVERING RADIOACTIVE MEDICAMENTS, A SHIELDED MEDICATION DELIVERY CASSETTE FOR RADIOACTIVE MEDICAMENTS AND A SHIELDED TUBING SET FOR ADMINISTRATION OF RADIOACTIVE MEDICAMENTS

(71) Applicant: SHL Medical AG, Zug (CH)

(72) Inventors: Christopher James Franzese, Boonton, NJ (US); Martin Michael Coyne, III, Boonton, NJ (US)

(21) Appl. No.: 19/109,666

(22) PCT Filed: Sep. 1, 2023
(86) PCT No.: PCT/EP2023/074073

§ 371 Date: Mar. 7, 2025

Related U.S. Application Data
(60) Provisional application No. 63/405,046, filed on Sep. 9, 2022.

(30) Foreign Application Priority Data
Mar. 24, 2023 (EP) 23163891.7

(51) Int. Cl. A61N 5/10; A61J 1/10; A61J 1/14; A61M 39/08; B32B 1/00; B32B 27/08; B32B 27/32; G21F 5/015

(52) U.S. Cl. CPC A61N 5/1007; A61J 1/10; A61J 1/1468; A61M 39/08; B32B 1/00; B32B 27/08; B32B 27/322; G21F 5/015; A61N 2005/1021; A61N 2005/1094; B32B 2307/714; B32B 2439/46

(57) ABSTRACT

The present disclosure relates to a shielded container for storing and/or transporting a radiopharmaceutical for delivery to a patient. The container includes an inner layer, a barrier layer disposed as an outer layer of the container, an intermediary tie layer disposed on the outside of the inner layer, and a shielding barrier layer disposed between the intermediary tie layer and the barrier layer.

### TECHNICAL FIELD

The present invention relates to a shielded flexible bag for storing and transporting a radiopharmaceutical, a shielded medication delivery cassette for containing a shielded flexible bag, and a shielded tubing set for administration of radiopharmaceutical to a patient.

### BACKGROUND

Radioisotopes are used for diagnostic (e.g., Positron Emission Tomography (PET) imaging) or therapeutic (treatment) purposes. For example, radioligand therapies (RLTs) are an emerging category for oncology treatment. RLTs deliver radiation to specifically targeted cancer cells by binding a ligand (finds cancer cells with a specific surface molecule) to a radioisotope (which emits radiation to kill the targeted cancer cell). Currently, RLTs are delivered by IV therapy. As they are radioactive agents, they are often manufactured just prior to use (due to short half-life) and supplied in vials which are shielded by a plexiglass case as shown in FIG. 1.

Administration of radioisotopes is complex due to the need to assemble administration components and provide appropriate shielding. FIG. 2 shows a representative intravenous therapy (IV) administration setup for an RLT with vial 8 containing the radioactive therapy vial within shielding element 9 being drawn out by suction from the pump 10 and diluted by saline 11. Although an RLT is shown in FIG. 2, nothing in this disclosure should be construed as precluding use with other radioisotopes or infusion setups.

### SUMMARY

The present invention has been made in view of the above problems and provides, inter alia, the following advantages.

According to a first embodiment, the invention relates to a shielded container for storing and/or transporting a radiopharmaceutical for delivery to a patient, the container comprising:
an inner layer;
a barrier layer disposed as an outer layer of the container;
an intermediary tie layer disposed on the outside of the inner layer; and
a shielding barrier layer disposed between the intermediary tie layer and the barrier layer.

According to a second embodiment, the invention relates to a shielded container, for storing and/or transporting a radiopharmaceutical for delivery to a patient, the container comprising:
an inner layer;
a shielding barrier layer disposed as an outer layer of the container,
an intermediary tie layer disposed on the outside of the inner layer; and
a barrier layer disposed between the intermediary tie layer and the shielding barrier layer.

Preferably, the shielded container according to the first or the second embodiment further comprises an additional intermediary tie layer disposed between the shielding barrier layer and the barrier layer.

Preferably, the shielded container according to the first or the second embodiment is a flexible bag for storing and transporting a radiopharmaceutical for delivery to a patient, and wherein the inner layer is an inner drug contact layer. Moreover, optionally, the inner drug contacting layer is made of cyclic olefin copolymer, COC, resin. Moreover, optionally, the barrier layer is made of polychlortrifluorethylene, PCTFE, material.

Preferably, the shielded container according to the first or the second embodiment is a shielded medication delivery cassette for containing a flexible bag or the previously mentioned shielded flexible bag. Preferably, the shielded medication cassette is usable with a pressure drive system that is configured to administrate diluted radiopharmaceutical from the flexible bag. Preferably, the inner layer is an inner cassette structural layer. Moreover, optionally, the shielded container further comprises an intermediary tie layer disposed between the inner cassette structural layer and the outer layer. Moreover, optionally, the inner cassette structural layer is made of one or more materials from a group of lead, metal, polycarbonate, PC, polyethylene terephthalate, PET, and/or polypropylene, PP. Moreover, optionally, the shielded container further comprises additional shielding material disposed on the outer shielding barrier layer.

### BRIEF DESCRIPTION OF THE DRAWINGS

FIG. 1 illustrates a conventional plexiglass case used for shielding and holding a vial containing radioactive agents.

FIG. 2 is a conventional IV administration setup for an RLT.

FIG. 3A is a layer structure of the container (bag).

FIGS. 3B-1 to 3B-3 illustrate various combinations of shielding layers for a shielded flexible bag according to Embodiment 1 of the present invention.

FIG. 4 is a shielded medication cassette for containing a shielded flexible bag, according to Embodiment 2 of the present invention.

FIG. 5 is a shielded tubing that is used for administration of radiopharmaceutical to a patient, according to Embodiment 3 of the present invention.

### DETAILED DESCRIPTION

The present invention is directed to embodiments that allow radioisotopes or radiopharmaceutical to be safely delivered to a patient while simplifying preparation and administration. One or more elements of the medication delivery system are provided with integral shielding elements which may be tailored to the expected types and/or intensity of radiation emitted by the medicament.

Embodiments 1 to 3 provided below illustrate different approaches to implement the described invention in various scenarios. Although different configurations of shielding are shown, the configurations shown here should be taken as being illustrative in nature and not limiting to the scope of the disclosure. Various aspects and configurations can be implemented as part of a single embodiment and there is no one “preferred” embodiment-instead, all three embodiments may be implemented in different preparation and administration situations:

Embodiment 1 shows a shielded flexible bag 100 that is advantageous when the shielding materials are relatively light and thin and may be bonded to the existing primary container materials.

Embodiment 2 shows a shielded medication cassette 201 that is advantageous when the shielding materials are relatively thick or heavy, are not conducive to bonding to the existing primary container materials.

Embodiment 3 shows a shielded tubing 300 that may be used with either or both of Embodiments 1 and 2 when maximum shielding is desired.

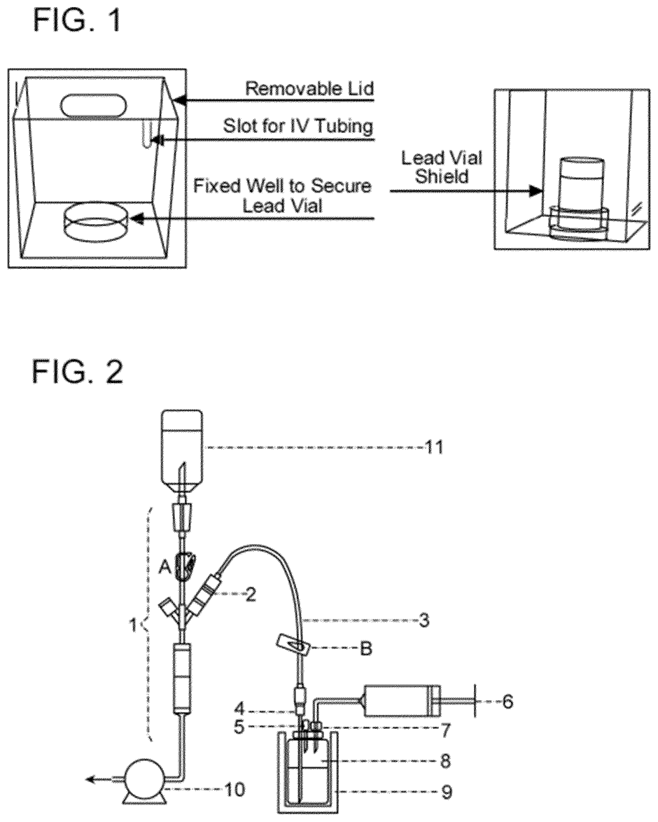

Clinically, Embodiments 1 and 2 allow delivery of medication that is already fully diluted, in contrast with the apparatus shown in FIGS. 1 and 2. During preparation, saline and a radioisotope are combined, and then the diluted radioisotope is filled directly into the flexible bag, which is then shielded as described herein for Embodiments 1 and 2. Such a system may further reduce radiation exposure compared to the status quo administration process, and maximally so with the addition of Embodiment 3.

### Embodiment 1—Shielded Flexible Bag

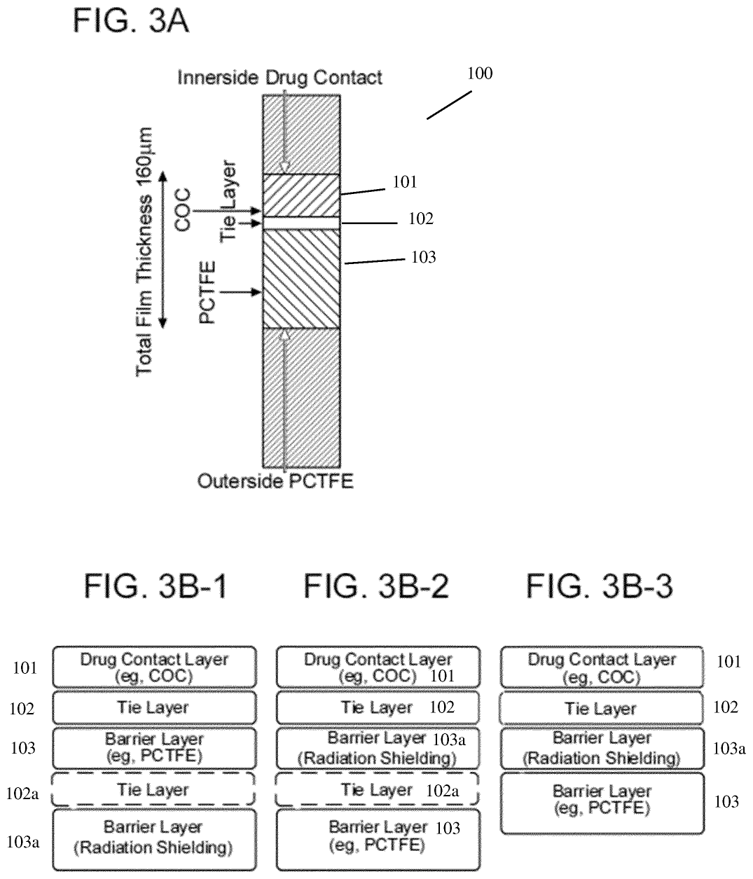

As seen in FIG. 3A illustrating a device (US 2020/0383869 A1), a flexible bag 100 is provided having a drug contacting layer 101 on the innerside and one or more barrier materials on the outerside, bonded through an intermediary tie layer 102. In the case of the device in US 2020/0383869 A1, the innerside drug contacting layer 101 is cyclic olefin copolymer (COC) and the outerside barrier layer 103 is Aclar Polychlorotrifluoroethylene (PCTFE) film. And there is at least a tie layer 102 disposed between the innerside drug contacting layer 101 is cyclic olefin copolymer (COC) and the outerside barrier layer 103 is Aclar Polychlorotrifluoroethylene (PCTFE) film. The thickness of the cyclic olefin copolymer (COC), Aclar Polychlorotrifluoroethylene (PCTFE) film or tie layer 102 can be varied depended on the flexibility of the multilayer films or the drugs stored in the bag 100. The bag 100 is provided for illustrative purposes, as is the description of barrier 103 and tie layers 102, but should not be construed as limiting the disclosure.

As seen in FIG. 3B, the present invention adds one or more shielding materials on the outerside layer 103. These materials are selected to prevent transmission of radiation from a medicament contained by the innerside drug contact layer 101 (made from COC for example) from being transmitted through the outerside layers 103. The shielding materials are shown in FIG. 3B-1 as placed on the outermost layer 103a of the outerside surface, but may be interposed between the tie layer 102 and barrier layer 103 (as seen in FIG. 3B-2); additional tie layers 102a may also be provided (e.g., between the outerside PCTFE and the radiation barriers). The additional tie layer 102a may also be omitted based on the moisture or oxygen transmission characteristics of the shielding materials, as seen in FIG. 3B-3. Selection of tie layers 102 and layer ordering may be based on the material characteristics, flexibility, desired bonding behaviors, manufacturing processes, or other considerations.

Embodiment 1 provides an integral shielded primary container 100 while preventing drug contact with the shielding materials. This is advantageous when the bag 100 is not surrounded by other materials that may appropriately provide shielding (e.g., the shielded cassette of Embodiment 2). Embodiment 1 is also advantageous when the shielding materials are relatively light and thin and may be bonded to the existing primary container materials. Embodiment 1 also allows any combination of materials to be used without impacting the drug contact or barrier properties of the primary container, which is advantageous if prior stability work has been completed on an unshielded container.

### Embodiment 2—Shielded Outer Medication Cassette

In certain instances, it may be infeasible to have a shielded bag 100 due to flexibility or lack of resilience of shielding materials, or it may be undesirable to have a shield bag 100 due to manufacturing inefficiencies of making both shielded and unshielded bag variants. In other instances, shielding required may preclude placement on the flexible bag 200 due to the thickness or materials required (i.e., cannot be bonded to a flexible material). In these instances, Embodiment 2 may be preferable, as it provides shielding in the cassette 201 containing the flexible bag 200 that is used to deliver the medication.

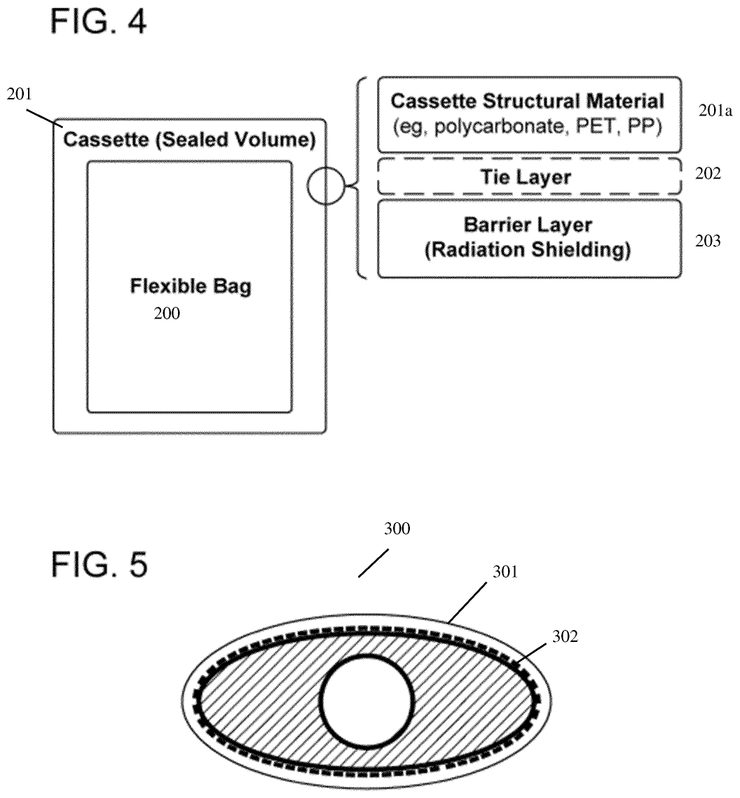

For instance, a pressure drive system (previously disclosed in FIG. 2) may be adapted for IV (vs. subcutaneous (SC)) delivery, using the same pumping mechanism and fluid reservoir, and changing the configuration of the cassette 201 and route of administration. FIG. 4 shows a pressure drive cassette 201 disclosed separately, with configuration of radiation shielding elements. Shielding materials 201a are selected for the inner cassette structural layer 201a and configured to avoid exposures to harmful radiation based on the isotope being administered. The cassette 201 may further comprise an intermediary tie layer 202 and an outer barrier layer 203. The intermediary tie layer 202 may be disposed between the inner cassette structural layer 201a and the outer barrier layer 203. Different shielding elements may be used separately in combination to prevent exposure to alpha, beta, or gamma particles emitted by the radioisotope and preventing exposure to harmful radiation. A cassette 201 configured as shown in FIG. 4 eliminates the need for the shielding shown in FIGS. 1 and 3B. For instance, combinations of lead, other metals, polycarbonate (PC), polyethylene terephthalate (PET) and/or polypropylene (PP) may be combined separately or in layers 201a in the cassette housing.

While the pressure drive system is shown in FIG. 2, Embodiment 2 may be used with any drive system where the flexible bag 200 is contained in an outer housing 201 that may be provided with shielding materials. Alternatively, some shielding materials (i.e., more flexible or bondable ones) may be provided on the bag 200, while others (i.e., more bulky or inflexible ones) may be provided in the cassette 201.

Any drive system may be used with the concept, the pressure drive system may be used advantageously to deliver a diluted isotope directly in a single flexible bag 200, eliminating the need for dilution or a separate saline bag (e.g., 11 in FIG. 2) and reducing the overall tubing length between the reservoir and patient (and thus, radiation exposure) while preserving the ability to stop, start, or throttle flow by manipulating the air injections introduced to the cassette, all as described previously. Such a system may further reduce radiation exposure compared to the status quo administration process.

### Embodiment 3—Shielded Tubing

Comparing Embodiments 1 and 2 to the status quo medication delivery setup of FIGS. 1 and 2, it is apparent that prior art and present invention do not provide shielding on the tubing set. To further reduce radiation exposure, the tubing set material can be shielded as well. In conjunction with either Embodiments 1 and 2, Embodiment 3 enables a completely shielded preparation and administration system, a substantial improvement over the apparatus of FIGS. 1 and 2.

FIG. 5 shows a tubing set 300 with outer shielding elements 301 and optional tie layer (dash) 302. This is particularly advantageous when an existing single or multi-lumen tubing set 300 is used, when the shielding materials are sufficiently flexible to accommodate movement of the tubing set 300 during administration, or when the shielding materials are compatible with manufacturing processes such as bonding or coextrusion.

The delivery devices described herein can be used for the treatment and/or prophylaxis of one or more of many different types of disorders.

Exemplary disorders include, but are not limited to: rheumatoid arthritis, inflammatory bowel diseases (e.g. Crohn's disease and ulcerative colitis), hypercholesterolaemia and/or dyslipidemia, cardiovascular disease, diabetes (e.g. type 1 or 2 diabetes), psoriasis, psoriatic arthritis, spondyloarthritis, hidradenitis suppurativa, Sjögren's syndrome, migraine, cluster headache, multiple sclerosis, neuromyelitis optica spectrum disorder, anaemia, thalassemia, paroxysmal nocturnal hemoglobinuria, hemolytic anaemia, hereditary angioedema, systemic lupus erythematosus, lupus nephritis, myasthenia gravis, Behçet's disease, hemophagocytic lymphohistiocytosis, atopic dermatitis, retinal diseases (e.g., age-related macular degeneration, diabetic macular edema), uveitis, infectious diseases, bone diseases (e.g., osteoporosis, osteopenia), asthma, chronic obstructive pulmonary disease, thyroid eye disease, nasal polyps, transplant, acute hypoglycaemia, obesity, anaphylaxis, allergies, sickle cell disease, Alzheimer's disease, Parkinson's disease, dementia with Lewy bodies, systemic infusion reactions, immunoglobulin E (IgE)-mediated hypersensitivity reactions, cytokine release syndrome, immune deficiencies (e.g., primary immunodeficiency, chronic inflammatory demyelinating polyneuropathy), enzyme deficiencies (e.g., Pompe disease, Fabry disease, Gaucher disease), growth factor deficiencies, hormone deficiencies, coagulation disorders (e.g., hemophilia, von Willebrand disease, Factor V Leiden), and cancer.

Exemplary types of drugs that could be included in the delivery devices described herein include, but are not limited to, small molecules, hormones, cytokines, blood products, enzymes, vaccines, anticoagulants, immunosuppressants, antibodies, antibody-drug conjugates, neutralizing antibodies, reversal agents, radioligand therapies, radioisotopes and/or nuclear medicines, diagnostic agents, bispecific antibodies, proteins, fusion proteins, peptibodies, polypeptides, pegylated proteins, protein fragments, nucleotides, protein analogues, protein variants, protein precursors, protein derivatives, chimeric antigen receptor T cell therapies, cell or gene therapies, oncolytic viruses, or immunotherapies.

Exemplary drugs that could be included in the delivery devices described herein include, but are not limited to, immuno-oncology or bio-oncology medications such as immune checkpoints, cytokines, chemokines, clusters of differentiation, interleukins, integrins, growth factors, coagulation factors, enzymes, enzyme inhibitors, retinoids, steroids, signaling proteins, pro-apoptotic proteins, anti-apoptotic proteins, T-cell receptors, B-cell receptors, or costimulatory proteins. Exemplary drugs that could be included in the delivery devices described herein include, but are not limited to, those exhibiting a proposed mechanism of action, such as human epidermal growth factor receptor 2 (HER-2) receptor modulators, interleukin (IL) modulators, interferon (IFN) modulators, complement modulators, glucagon-like peptide-1 (GLP-1) modulators, glucose-dependent insulinotropic polypeptide (GIP) modulators, cluster of differentiation 38 (CD38) modulators, cluster of differentiation 22 (CD22) modulators, C1 esterase modulators, bradykinin modulators, C—C chemokine receptor type 4 (CCR4) modulators, vascular endothelial growth factor (VEGF) modulators, B-cell activating factor (BAFF), P-selectin modulators, neonatal Fc receptor (FcRn) modulators, calcitonin gene-related peptide (CGRP) modulators, epidermal growth factor receptor (EGFR) modulators, cluster of differentiation 79B (CD79B) modulators, tumor-associated calcium signal transducer 2 (Trop-2) modulators, cluster of differentiation 52 (CD52) modulators, B-cell maturation antigen (BCMA) modulators, enzyme modulators, platelet-derived growth factor receptor A (PDGFRA) modulators, cluster of differentiation 319 (CD319 or SLAMF7) modulators, programmed cell death protein 1 and programmed death-ligand 1 (PD-1/PD-L1) inhibitors/modulators, B-lymphocyte antigen cluster of differentiation 19 (CD19) inhibitors, B-lymphocyte antigen cluster of differentiation 20 (CD20) modulators, cluster of differentiation 3 (CD3) modulators, cytotoxic T-lymphocyte-associated protein 4 (CTLA-4) inhibitors, T-cell immunoglobulin and mucin-domain containing-3 (TIM-3) modulators, T cell immunoreceptor with Ig and ITIM domains (TIGIT) modulators, V-domain Ig suppressor of T cell activation (VISTA) modulators, indoleamine 2,3-dioxygenase (IDO or INDO) modulators, poliovirus receptor-related immunoglobulin domain-containing protein (PVRIG) modulators, lymphocyte-activation gene 3 (LAG3; also known as cluster of differentiation 223 or CD223) antagonists, cluster of differentiation 276 (CD276 or B7-H3) antigen modulators, cluster of differentiation 47 (CD47) antagonists, cluster of differentiation 30 (CD30) modulators, cluster of differentiation 73 (CD73) modulators, cluster of differentiation 66 (CD66) modulators, cluster of differentiation W137 (CDw137) agonists, cluster of differentiation 158 (CD158) modulators, cluster of differentiation 27 (CD27) modulators, cluster of differentiation 58 (CD58) modulators, cluster of differentiation 80 (CD80) modulators, cluster of differentiation 33 (CD33) modulators, cluster of differentiation 159 (CD159 or NKG2) modulators, glucocorticoid-induced TNFR-related (GITR) protein modulators, Killer Ig-like receptor (KIR) modulators, growth arrest-specific protein 6 (GAS6)/AXL pathway modulators, A proliferation-inducing ligand (APRIL) receptor modulators, human leukocyte antigen (HLA) modulators, epidermal growth factor receptor (EGFR) modulators, B-lymphocyte cell adhesion molecule modulators, cluster of differentiation w123 (CDw123) modulators, Erbb2 tyrosine kinase receptor modulators, endoglin modulators, mucin modulators, mesothelin modulators, hepatitis A virus cellular receptor 2 (HAVCR2) antagonists, cancer-testis antigen (CTA) modulators, tumor necrosis factor receptor superfamily, member 4 (TNFRSF4 or OX40) modulators, adenosine receptor modulators, inducible T cell co-stimulator (ICOS) modulators, cluster of differentiation 40 (CD40) modulators, tumor-infiltrating lymphocytes (TIL) therapies, or T-cell receptor (TCR) therapies. Exemplary drugs that could be included in the delivery devices described herein include, but are not limited to: etanercept, abatacept, adalimumab, evolocumab, exenatide, secukinumab, erenumab, galcanezumab, fremanezumab-vfrm, alirocumab, methotrexate (amethopterin), tocilizumab, interferon beta-1a, interferon beta-1b, peginterferon beta-1a, sumatriptan, darbepoetin alfa, belimumab, sarilumab, semaglutide, dupilumab, reslizumab, omalizumab, glucagon, epinephrine, naloxone, insulin, amylin, vedolizumab, eculizumab, ravulizumab, crizanlizumab-tmca, certolizumab pegol, satralizumab, denosumab, romosozumab, benralizumab, emicizumab, tildrakizumab, ocrelizumab, ofatumumab, natalizumab, mepolizumab, risankizumab-rzaa, ixekizumab, and immune globulins.

Exemplary drugs that could be included in the delivery devices described herein may also include, but are not limited to, oncology treatments such as ipilimumab, nivolumab, pembrolizumab, atezolizumab, durvalumab, avelumab, cemiplimab, rituximab, trastuzumab, ado-trastuzumab emtansine, fam-trastuzumab deruxtecan-nxki, pertuzumab, transtuzumab-pertuzumab, alemtuzumab, belantamab mafodotin-blmf, bevacizumab, blinatumomab, brentuximab vedotin, cetuximab, daratumumab, elotuzumab, gemtuzumab ozogamicin, 90-Yttrium-ibritumomab tiuxetan, isatuximab, mogamulizumab, moxetumomab pasudotox, obinutuzumab, ofatumumab, olaratumab, panitumumab, polatuzumab vedotin, ramucirumab, sacituzumab govitecan, tafasitamab, or margetuximab.

Exemplary drugs that could be included in the delivery devices described herein include “generic” or biosimilar equivalents of any of the foregoing, and the foregoing molecular names should not be construed as limiting to the “innovator” or “branded” version of each, as in the non-limiting example of innovator medicament adalimumab and biosimilars such as adalimumab-afzb, adalimumab-atto, adalimumab-adbm, and adalimumab-adaz.

Exemplary drugs that could be included in the delivery devices described herein also include, but are not limited to, those used for adjuvant or neoadjuvant chemotherapy, such as an alkylating agent, plant alkaloid, antitumor antibiotic, antimetabolite, or topoisomerase inhibitor, enzyme, retinoid, or corticosteroid. Exemplary chemotherapy drugs include, by way of example but not limitation, 5-fluorouracil, cisplatin, carboplatin, oxaliplatin, doxorubicin, daunorubicin, idarubicin, epirubicin, paclitaxel, docetaxel, cyclophosphamide, ifosfamide, azacitidine, decitabine, bendamustine, bleomycin, bortezomib, busulfan, cabazitaxel, carmustine, cladribine, cytarabine, dacarbazine, etoposide, fludarabine, gemcitabine, irinotecan, leucovorin, melphalan, methotrexate, pemetrexed, mitomycin, mitoxantrone, temsirolimus, topotecan, valrubicin, vincristine, vinblastine, or vinorelbine.

Exemplary drugs that could be included in the delivery devices described herein also include, but are not limited to, analgesics (e.g., acetaminophen), antipyretics, corticosteroids (e.g. hydrocortisone, dexamethasone, or methylprednisolone), antihistamines (e.g., diphenhydramine or famotidine), antiemetics (e.g., ondansetron), antibiotics, antiseptics, anticoagulants, fibrinolytics (e.g., recombinant tissue plasminogen activator [r-TPA]), antithrombolytics, or diluents such as sterile water for injection (SWFI), 0.9% Normal Saline, 0.45% normal saline, 5% dextrose in water, 5% dextrose in 0.45% normal saline, Lactated Ringer's solution, Heparin Lock Flush solution, 100 U/mL Heparin Lock Flush Solution, or 5000 U/mL Heparin Lock Flush Solution.

Pharmaceutical formulations including, but not limited to, any drug described herein are also contemplated for use in the delivery devices described herein, for example pharmaceutical formulations comprising a drug as listed herein (or a pharmaceutically acceptable salt of the drug) and a pharmaceutically acceptable carrier. Such formulations may include one or more other active ingredients (e.g., as a combination of one or more active drugs), or may be the only active ingredient present, and may also include separately administered or co-formulated dispersion enhancers (e.g. an animal-derived, human-derived, or recombinant hyaluronidase enzyme), concentration modifiers or enhancers, stabilizers, buffers, or other excipients.

Exemplary drugs that could be included in the delivery devices described herein include, but are not limited to, a multi-medication treatment regimen such as AC, Dose-Dense AC, TCH, GT, EC, TAC, TC, TCHP, CMF, FOLFOX, mFOLFOX6, mFOLFOX7, FOLFCIS, CapeOx, FLOT, DCF, FOLFIRI, FOLFIRINOX, FOLFOXIRI, IROX, CHOP, R-CHOP, RCHOP-21, Mini-CHOP, Maxi-CHOP, VR-CAP, Dose-Dense CHOP, EPOCH, Dose-Adjusted EPOCH, R-EPOCH, CODOX-M, IVAC, HyperCVAD, R-HyperCVAD, SC-EPOCH-RR, DHAP, ESHAP, GDP, ICE, MINE, CEPP, CDOP, GemOx, CEOP, CEPP, CHOEP, CHP, GCVP, DHAX, CALGB 8811, HIDAC, MOPAD, 7+3, 5+2, 7+4, MEC, CVP, RBAC500, DHA-Cis, DHA-Ca, DHA-Ox, RCVP, RCEPP, RCEOP, CMV, DDMVAC, GemFLP, ITP, VIDE, VDC, VAI, VDC-IE, MAP, PCV, FCR, FR, PCR, HDMP, OFAR, EMA/CO, EMA/EP, EP/EMA, TP/TE, BEP, TIP, VIP, TPEx, ABVD, BEACOPP, AVD, Mini-BEAM, IGEV, C-MOPP, GCD, GEMOX, CAV, DT-PACE, VTD-PACE, DCEP, ATG, VAC, VeIP, OFF, GTX, CAV, AD, MAID, AIM, VAC-IE, ADOC, or PE.

1-13. (canceled)

14. A shielded container for storing and/or transporting a radiopharmaceutical for delivery to a patient, the container comprising:
an inner layer;
a barrier layer disposed as an outer layer of the container;
an intermediary tie layer disposed on the outside of the inner layer; and
a shielding barrier layer disposed between the intermediary tie layer and the barrier layer.

15. The shielded container of claim 14, further comprising an additional intermediary tie layer disposed between the shielding barrier layer and the barrier layer.

16. The shielded container of claim 14, wherein the shielded container is a shielded flexible bag for storing and transporting a radiopharmaceutical for delivery to a patient, and wherein the inner layer is an inner drug contacting layer.

17. The shielded container of claim 16, wherein the inner drug contacting layer is made of cyclic olefin copolymer, COC, resin.

18. The shielded container of claim 16, wherein the barrier layer is made of polychlortrifluorethylene, PCTFE, material.

19. The shielded container of claim 16, wherein the shielded medication cassette is usable with a pressure drive system that is configured to administrate diluted radiopharmaceutical from the flexible bag, and wherein the inner layer is an inner cassette structural layer.

20. The shielded container of claim 18, wherein the inner cassette structural layer is made of one or more materials from a group of lead, metal, polycarbonate, PC, polyethylene terephthalate, PET, and/or polypropylene, PP.

21. The shielded container of claim 16, further comprising additional shielding material disposed on the outer barrier layer.

22. A shielded container for storing and/or transporting a radiopharmaceutical for delivery to a patient, the container comprising:
an inner layer;
a shielding barrier layer disposed as an outer layer of the container;
an intermediary tie layer disposed on the outside of the inner layer; and
a barrier layer disposed between the intermediary tie layer and the shielding barrier layer.

23. The shielded container of claim 22, further comprising an additional intermediary tie layer disposed between the shielding barrier layer and the barrier layer.

24. The shielded container of claim 22, wherein the shielded container is a shielded flexible bag for storing and transporting a radiopharmaceutical for delivery to a patient, and wherein the inner layer is an inner drug contacting layer.

25. The shielded container of claim 24, wherein the inner drug contacting layer is made of cyclic olefin copolymer, COC, resin.

26. The shielded container of claim 24, wherein the barrier layer is made of polychlortrifluorethylene, PCTFE, material.

27. The shielded container of claim 24, wherein the shielded medication cassette is usable with a pressure drive system that is configured to administrate diluted radiopharmaceutical from the flexible bag, and wherein the inner layer is an inner cassette structural layer.

28. The shielded container of claim 27, wherein the inner cassette structural layer is made of one or more materials from a group of lead, metal, polycarbonate, PC, polyethylene terephthalate, PET, and/or polypropylene, PP.

29. The shielded container of claim 24, further comprising additional shielding material disposed on the outer barrier layer.

30. A shielded tubing set for administration of radiopharmaceutical to a patient, the shielded tubing set fluidly connectable to the shielded container of claim 14, wherein the shielded tubing set comprises:
a tubing; and
outer shielding elements at the outer surface of the tubing.

31. The shielded tubing set of claim 30, wherein the shielding elements form a contiguous shielding layer at least a portion of the circumferential surface of the tubing.

32. The shielded tubing set of claim 30, further comprising:
an inner tie layer.
